(19) United States
(12) Patent Application Publication
(10) Pub. No.: US 2024/0185985 A1
(43) Pub. Date: Jun. 6, 2024

(54) GENETIC FOOD SCORING SYSTEM

(71) Applicant: Robert Giardini, Franklin, MA (US)

(72) Inventor: Robert Giardini, Franklin, MA (US)

(21) Appl. No.: 18/550,792

(22) PCT Filed: Mar. 16, 2022
(86) PCT No.: PCT/US22/20653

§ 371 Date: Sep. 15, 2023

Related U.S. Application Data
(60) Provisional application No. 63/161,517, filed on Mar. 16, 2021.

(51) Int. Cl. G16H 20/60; G16H 10/60

(52) U.S. Cl. CPC G16H 20/60; G16H 10/60

(57) ABSTRACT

In accordance with embodiments of the invention, a food scoring system is provided. A centralized data processing server is provided and hosts a database of nutrition information of foods and genetic testing information of a patient. The genetic testing information includes recommended consumption of macronutrients and levels of micronutrients based on the patient's genetic composition. A software application is operable for the patient to enter food consumed or food to be consumed into the application. The software application communicates with the centralized data process server. By operation of the processing server, the efficacy of the nutrition of the meal relative to the patient's recommended intake of macronutrients and micronutrients is analyzed. By operation of the processing server, a meal rating score and a micronutrient rating score are calculated based on the nutrition of the meal. The meal rating score and the micronutrient score are displayed via the software application.

### BACKGROUND OF THE INVENTION

### I. CROSS-REFERENCE TO RELATED APPLICATIONS

This application claims priority to International Patent Application No. PCT/US22/20653, filed Mar. 16, 2022, which claims priority to U.S. Provisional Patent Application Ser. No. 63/161,517, filed Mar. 16, 2021, the entirety of which are incorporated by reference as if fully disclosed herein.

### II. Field of the Invention

The present invention relates generally to a system for using the macronutrient and micronutrient ratings of the genotype of an individual to plan efficient meals.

### III. General Background

For as long as humans have been eating, they have been interested in dieting and weight loss. Popular diets often target generalized approaches, such as reducing fat or carbohydrates. However, these approaches to dieting and healthy eating are not tailored to the individual needs of each person. For example, a low carbohydrate diet may work well for one person but not another because of the variations in genotypes between individuals. Accordingly, what is needed is a system utilizing the unique genotype of a person to plan efficient meals.

### SUMMARY OF THE INVENTION

In accordance with embodiments of the invention, a food scoring system is provided. The system includes a centralized data processing server. A database of nutrition information of foods is hosted on the centralized data processing server. The genetic testing information of a patient is received and stored on the centralized data processing server. The genetic testing information includes recommended consumption of macronutrients and levels of micronutrients based on the genetic composition of the patient. The system includes a software application operable for the patient to enter food consumed or food to be consumed as a meal into the application. The software application communicates with the centralized data process server. By operation of the centralized data processing server, the efficacy of the nutrition of the meal relative to the patient's recommended intake of macronutrients and micronutrients is analyzed. By operation of the centralized data processing server, a meal rating score and a micronutrient rating score are calculated based on the nutrition of the meal. The meal rating score and the micronutrient score are displayed via the software application.

In accordance with embodiments of the invention, a food scoring system is provided. The system includes a centralized data processing server. A database of nutrition information of foods is hosted on the centralized data processing server. The genetic testing information of each patient in a population of patients is received and stored on the centralized data processing server. The genetic testing information includes recommended consumption of macronutrients and levels of micronutrients based on the genetic composition of each patient in the population of patients. The system includes a software application operable for each patient in the population of patients to enter food consumed or food to be consumed as a meal into the application. The software application communicates with the centralized data process server. By operation of the centralized data processing server, the efficacy of the nutrition of the meal relative to each patient in the population of patients recommended intake of macronutrients and micronutrients is analyzed. By operation of the centralized data processing server, a meal rating score and a micronutrient rating score are calculated based on the nutrition of the meal. The meal rating score and the micronutrient score are displayed via the software application.

In accordance with embodiments of the invention, a food scoring system is provided. The system includes a centralized data processing server, a database of nutrition information of foods and a software application in communication with the centralized data processing server. The patient enters food consumed or food to be consumed as a meal into the application. The genetic testing information including recommended consumption of macronutrients and levels of micronutrients based on the genetic composition of the patient is uploaded to the centralized data processing server. By operation of the centralized data processing server, the nutrition of the meal relative to the patient's recommended intake of macronutrients and micronutrients is analyzed. By operation of the centralized data processing server, a meal rating score and a micronutrient rating score are calculated based on the nutrition of the meal. The meal rating score and the micronutrient score are displayed via the software application.

In accordance with embodiments of the invention, a method for food scoring is provided. The method includes a first step of providing a centralized data processing server. The method includes a second step of hosting a database of nutrition information of foods on the centralized data processing server. The method includes a third step of receiving and storing genetic testing information of each patient in a population of patients in the centralized data processing server, wherein the genetic testing information includes recommended consumption of macronutrients and levels of micronutrients based on the genetic composition of each patient in the population of patients. The method includes a fourth step of providing a software application operable for each patient in the population of patients to enter food consumed or food to be consumed as a meal into the application. The software application communicates with the centralized data process server. The method includes a fifth step of analyzing, by operation of the centralized data processing server, the efficacy of the nutrition of the meal relative to the recommended intake of macronutrients and micronutrients of each patient in the population of patients. The method includes a sixth step of calculating, by operation of the centralized data processing server, a meal rating score and a micronutrient rating score based on the nutrition of the meal. The method includes a seventh step of displaying the meal rating score and the micronutrient score via the software application.

### BRIEF DESCRIPTION OF THE DRAWINGS

The foregoing and other objects, features, and advantages of the invention are apparent from the following detailed description taken in conjunction with the accompanying drawings in which like parts are given like reference numerals and, wherein:

FIG. 1 illustrates a plan view of a genetic food scoring system in accordance with embodiments of the invention.

FIG. 2 illustrates a patient input of a food to be consumed into a genetic food scoring system in accordance with embodiments of the invention.

FIG. 3 illustrates a patient input of a food to be consumed into a genetic food scoring system in accordance with embodiments of the invention.

FIG. 4 illustrates a genetic food scoring system log of foods consumed by a patient in accordance with embodiments of the invention.

FIG. 5 illustrates a genetic food scoring system displaying a meal rating in accordance with embodiments of the invention.

FIG. 6 illustrates a genetic food scoring system displaying meal rating levels in accordance with embodiments of the invention.

FIG. 7 illustrates a genetic food scoring system log of foods consumed by a patient in accordance with embodiments of the invention.

FIG. 8 illustrates a genetic food scoring system displaying a meal rating in accordance with embodiments of the invention.

FIG. 9 illustrates a genetic food scoring system displaying a meal rating in accordance with embodiments of the invention.

FIG. 10 illustrates a genetic food scoring system displaying a meal rating in accordance with embodiments of the invention.

FIG. 11 illustrates a genetic food scoring system displaying a meal rating in accordance with embodiments of the invention.

FIG. 12 illustrates a genetic food scoring system displaying a weekly report in accordance with embodiments of the invention.

FIG. 13 illustrates a genetic food scoring system displaying a weekly report for a micronutrient in accordance with embodiments of the invention.

FIG. 14 illustrates a genetic food scoring system displaying a weekly report for a micronutrient in accordance with embodiments of the invention.

FIG. 15 illustrates a genetic food scoring system displaying a recommended list of foods in accordance with embodiments of the invention.

FIG. 16 illustrates a flow chart of a process of a genetic food scoring system in accordance with embodiments of the invention.

The images in the drawings are simplified for illustrative purposes and are not depicted to scale. Within the descriptions of the figures, similar elements are provided similar names and reference numerals as those of the previous figure(s). The specific numerals assigned to the elements are provided solely to aid in the description and are not meant to imply any limitations (structural or functional) on the invention.

The appended drawings illustrate exemplary configurations of the invention and, as such, should not be considered as limiting the scope of the invention that may admit to other equally effective configurations. It is contemplated that features of one configuration may be beneficially incorporated in other configurations without further recitation.

### DETAILED DESCRIPTION

The embodiments of the disclosure will be best understood by reference to the drawings, wherein like parts are designated by like numerals throughout. It will be readily understood that the components, as generally described and illustrated in the Figures herein, could be arranged and designed in a wide variety of different configurations or be entirely separate. Thus, the following more detailed description of the embodiments of the system and method of the disclosure, as represented in the Figures is not intended to limit the scope of the disclosure, as claimed, but is merely representative of possible embodiments of the disclosure.

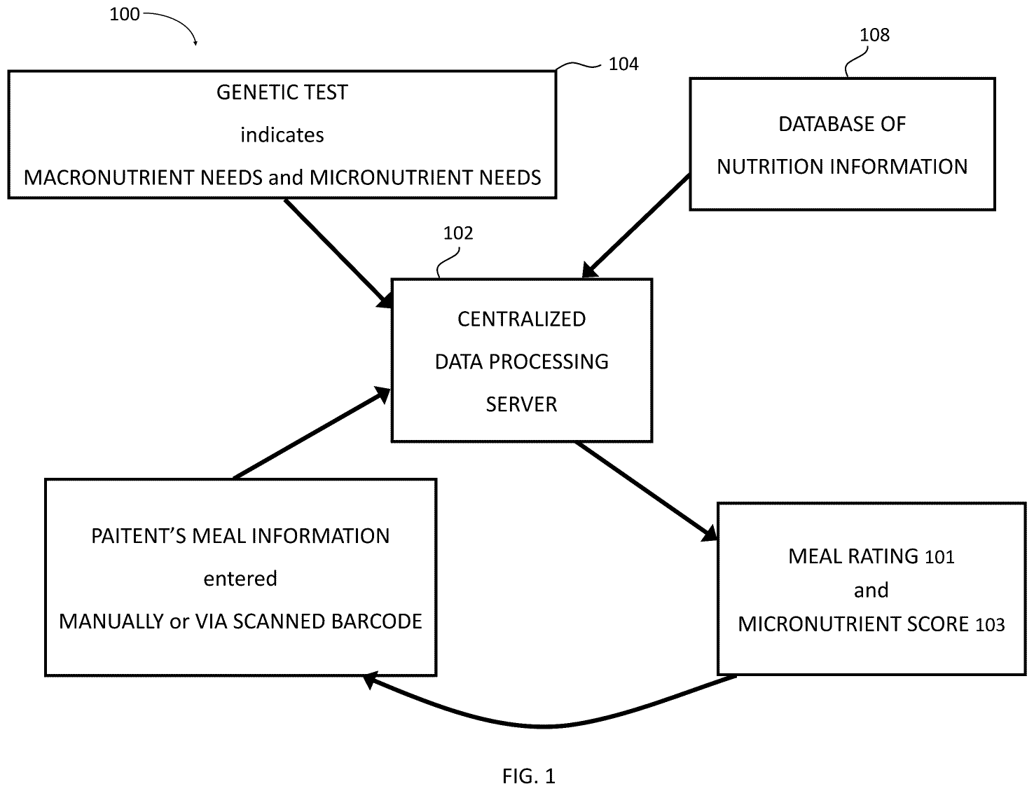

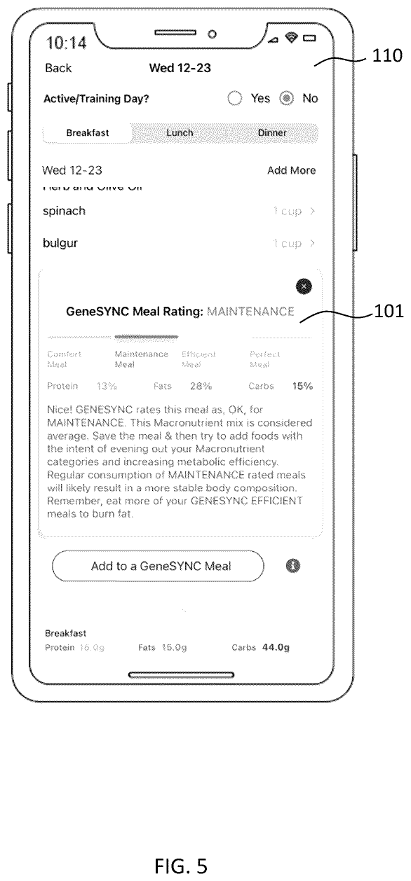

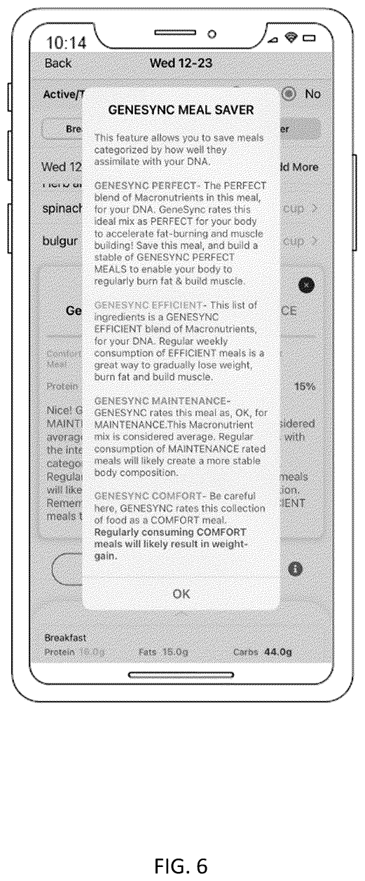

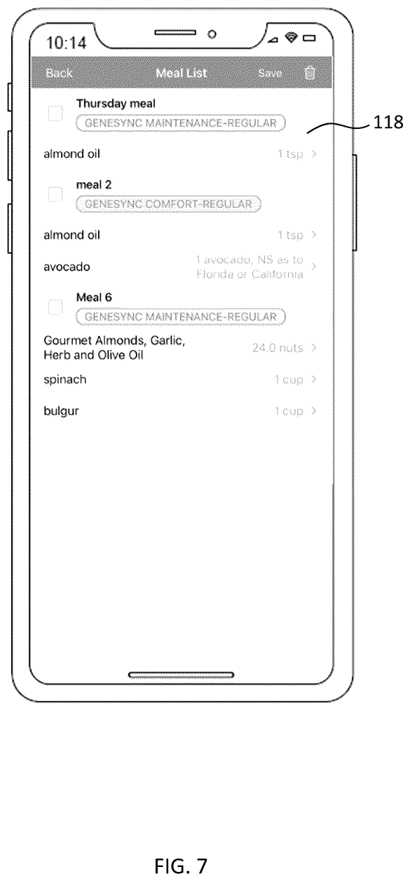

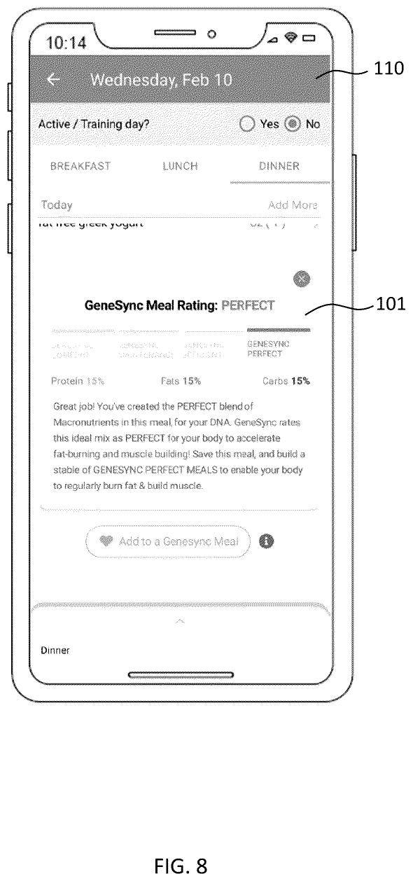

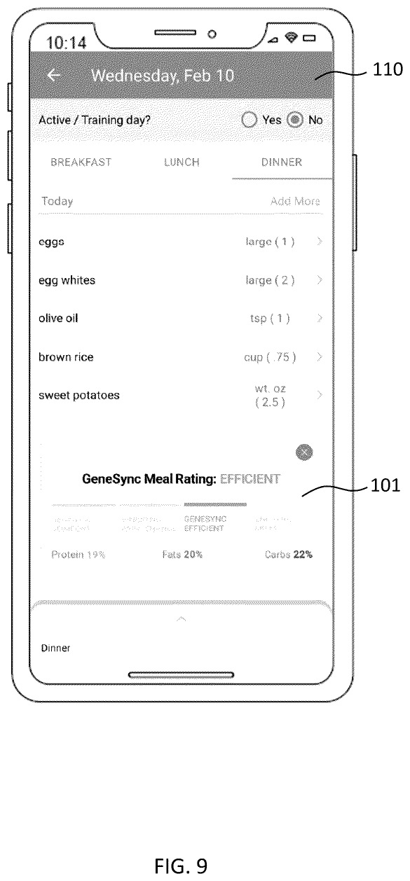

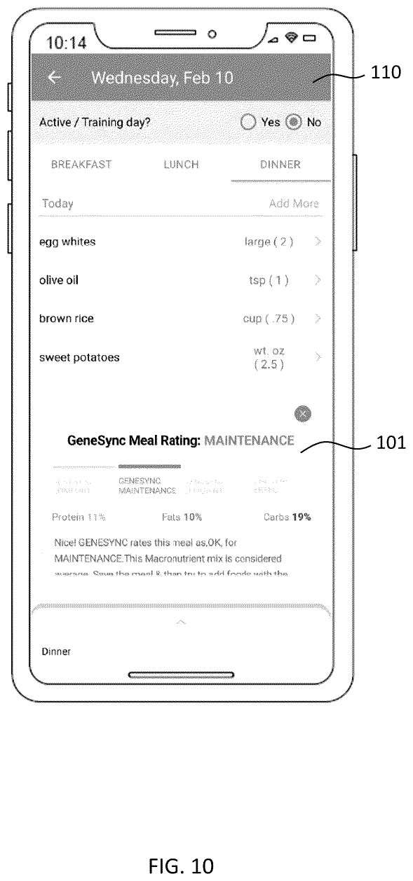

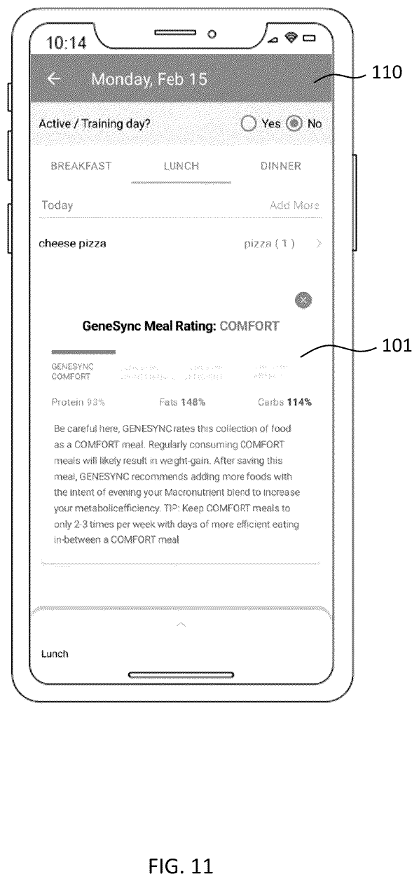

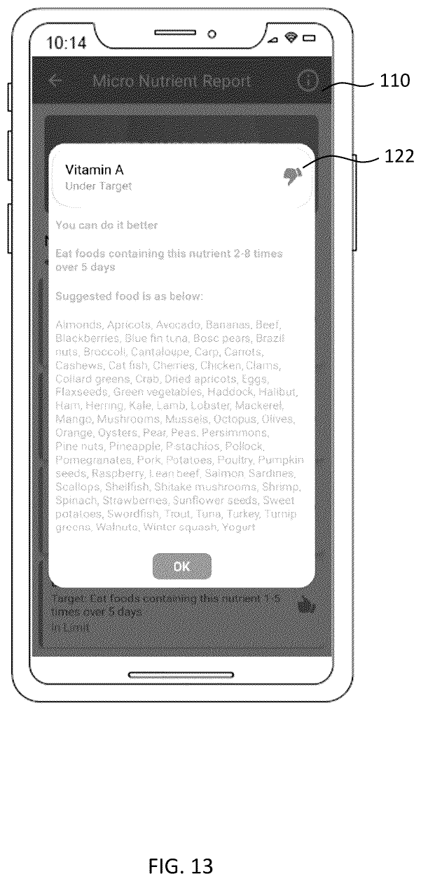

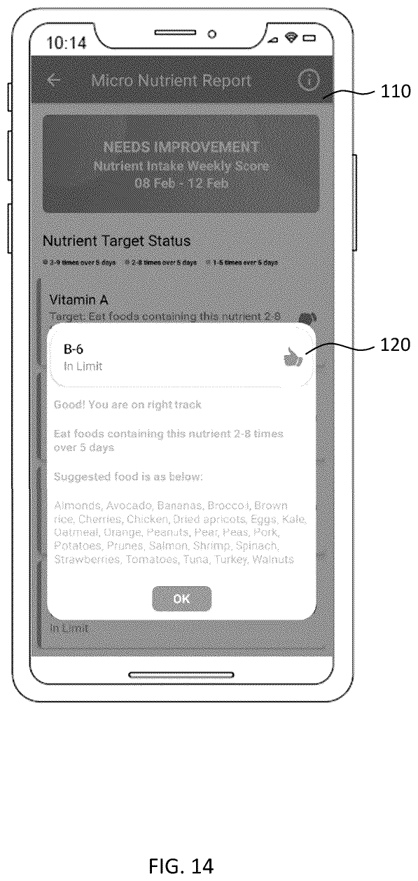

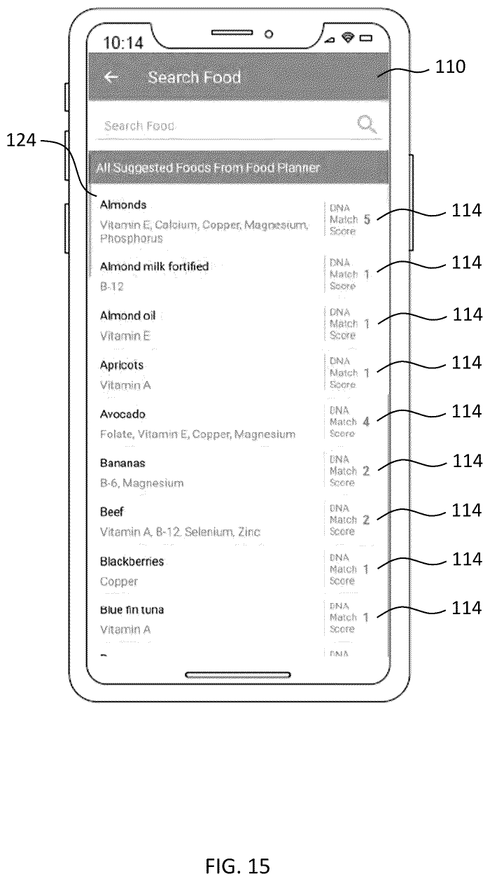

In accordance with embodiments of the invention, as illustrated in FIGS. 1-16, a genetic food scoring system 100 is provided. An object of the invention is to enable patients to plan meals uniquely tailored to their individual genetic compositions to maximize weight loss and healthy eating. Another object of the invention is to enable professionals, such as personal trainers, dieticians, and health programs, to manage individualized weight loss plans for many patients simultaneously because each patient has a unique DNA structure. As illustrated in FIG. 1, the genetic food scoring system 100 includes providing a centralized data processing server 102. The genetic food scoring system 100 utilizes genetic testing results 104, such as a DNA report, of a patient 106 to generate a meal rating 101 and a micronutrient score 103 for efficacy of a meal for nutrition, healthy eating, and weight loss. A database 108 of nutrition information of foods is hosted on the centralized data processing server 102. Alternatively, the database 108 of nutrition information may be hosted by a third party and accessed by the centralized data processing server 102. The genetic testing results 104 of the patient 106 are received and stored on the centralized data processing server 102. Alternatively, the genetic testing results 104 may be hosted by a third party and accessed by the centralized data processing server 102. The system 100 is operable to host the genetic testing results 104 of each patient 106 in a population of patients 107, which is advantageous for trainers and healthcare providers, for example, to manage personalized diets for each patient 106 of a population of patients 107. The genetic testing results 104 include recommended consumption of macronutrients and levels of micronutrients based on the genetic composition of patient 106, for example. The system 100 includes providing a software application 110, such as a website or mobile application, operable for the patient 106 to enter food 112 consumed or to be consumed as a meal into the software application 110. The patient 106 may manually enter the foods to be consumed or scan a barcode of a product. The database 108 of nutrition information of foods includes the nutrition information of foods and the specific nutrition information of foods sold with barcodes. The software application 110 communicates with the centralized data processing server 102 to upload the contents of a meal entered by the patient 106. The efficacy of the nutrition of the meal relative to the recommended intake of macronutrients and micronutrients of the patient 106 is analyzed by operation of the centralized data processing server 102 by comparing nutritional information of components of a meal to the database 108 of nutrition information. A meal rating 101 and a micronutrient score 103 based on the nutrition of the meal are calculated by operation of the centralized data processing server 102. The meal rating 101 and the micronutrient score 103 are displayed via the software application 110, for example. A patient 106 may choose to modify the meal to be consumed to increase the meal rating 101 and a micronutrient score 103. In conjunction with the centralized data processing server 102, the software application 110 is operable to keep a log 118 of the foods consumed by the patient 106 to enable the patient 106 to keep track of the efficacy of meals for a period of time, such as a week, for example.

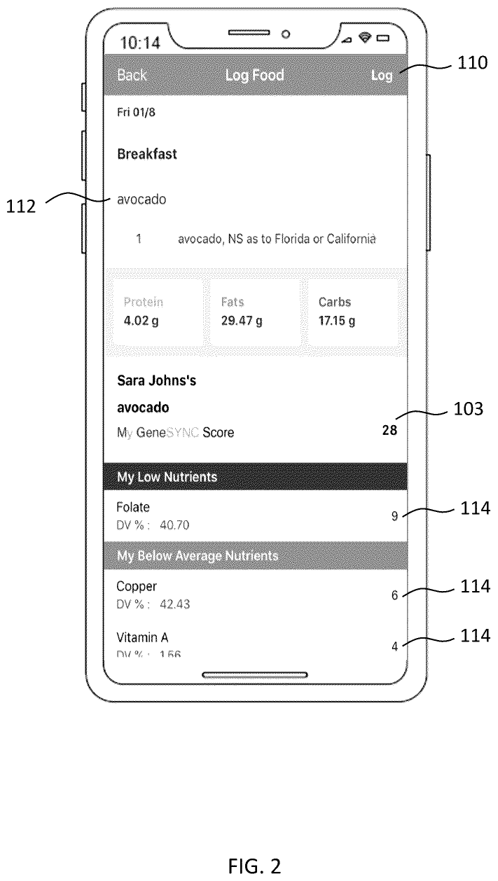

The genetic food scoring system 100 may be mobile application 110 based, as illustrated in FIGS. 2-15, to enable a patient 106 to log foods 112 consumed or intended to be consumed to determine the efficacy of the meal relative to the DNA report 104 of the patient 106. The user may log food into the mobile application 110 by manual entry (FIGS. 2-4) or by scanning a barcode, or selecting foods from a website if the system is integrated into a website to order food, such as a grocery store, online grocery delivery service, or restaurant, for example. By scanning a barcode via the mobile application 110, the centralized processing server 102 is operable to retrieve the nutrition information for the food from the database of nutrition information of foods and calculate both a meal rating 101 and a micronutrient score 103.

In one embodiment, the genetic food scoring system 100 rates meals based on two sets of criteria—macronutrients and micronutrients. Macronutrients are typically divided into three categories: lean protein, healthy fats, and complex carbohydrates. A typical recommended listing of micronutrients is Vitamin A, Vitamin B6, Folate, Vitamin B12, Vitamin C, Vitamin D, Vitamin E, Dietary Choline, Calcium, Copper, Iron, Magnesium, Phosphorus, Selenium, and Zinc. An exemplary DNA report 104 of a patient 106 is illustrated in Table 1 below. The recommended daily food consumption of the patient 106 with respect to macronutrients, the daily intake of lean protein is recommended to be 20-25%, the daily intake of healthy fats is recommended to be 25-30%, and the daily intake of complex carbohydrates is 50-60%. Exemplary sources of lean protein include beans, legumes, nuts, seeds, whole grains and vegetables. These ranges vary among patients as a result of their individual genetic composition. The system 100 is operable to calculate the efficiency of meals for each patient 106 based on the individual ranges of each category of macronutrients.

TABLE 1
Exemplary Macronutrient Listing of a DNA Report.

| | | Percent of Daily |
|---|---|---|
| Food | Recommendation | Consumption |
| Lean Protein | Choose a reduced-calorie diet | 20%-25% |
| | that is between 20-25% protein. | |
| | Get your protein from mostly | |
| | plant food sources such as | |
| | beans, legumes, nuts, seeds, | |
| | whole grains and vegetables. | |
| Healthy Fats | Choose either a low- or moderate- | 25-35% |
| | fat, reduced-calorie diet. Get | |
| | your fats mostly from plant foods, | |
| | but avoid excess added oils. | |
| Complex | Choose a plant-based diet that | 50%-60% |
| Carbohydrates | is high in complex carbs (veggies, | |
| | beans, whole grains, etc.), and | |
| | avoid simple or processed carbs | |
| | (fries, chips, crackers, etc.). | |

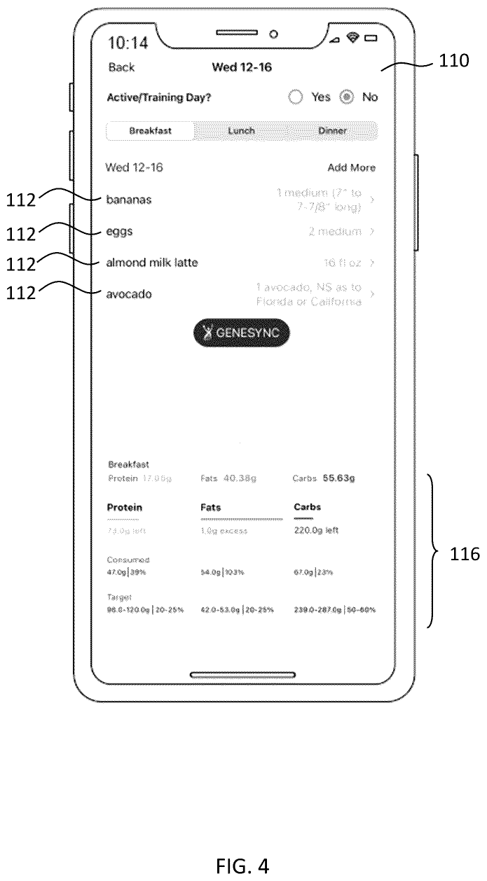

To calculate a meal rating 101, a patient obtains a DNA test 104 from any DNA testing lab that includes the ideal recommendations for macronutrients and lists micronutrient tendencies. The results of the DNA test 104 are uploaded to the centralized data processing server 102 via the software application 110, manual entry, or uploading through any interface operable to import the test results. In the example in included in Table 1, the recommended daily max total of macronutrients is 20%-25% of lean protein, 25%-35% of healthy fats, and 50%-60% of complex carbohydrates. A log is created to journal to how close food consumed is to the exact blend of daily recommended macronutrients. By operation of the centralized data process server 102, a patient 106 is guided towards their ideal macronutrient blend. To assist with comprehension and guidance, a meal rating score 101 may be generated to inform the patient 106 of the efficacy of a meal with respect to their recommended intake of macronutrients. As illustrated in FIG. 4, the software application 110 is operable to show a breakdown 116 of foods consumed, target amount, and how much left of each macronutrient category to go for a time period, such as a day.

In order to enable patients 106 to understand the efficacy of meals consumed, the system 100 calculates meal ratings 101 to inform the user. An exemplary method of calculating meal ratings 101 categorizes meals as perfect, efficient, maintenance, and comfort, as illustrated in FIGS. 2-8. This helps the patient create an intake evenness and overall macronutrient efficiency consistent with their DNA. In this embodiment, a meal rating 101 is calculated based upon the rate of which each of the macronutrients daily total is being consumed. The closer the rate of consumption of each macronutrient category is equal to that of all other macronutrient rate of consumption in daily percent consumed, the higher that meal rating 101 will be. A perfect meal rating is a meal that has the blend of macronutrients recommended for the patient 106 as indicated in the DNA report 104. For a perfect meal, the blend accelerates fat burning and muscle building. This is the most efficient strategy for the patient 106 as each macronutrient is being loaded into the body at an even and equal pace. The next most effective meal rating 101 is an efficient meal. An efficient meal is a meal where the difference of consumed protein and carbohydrates are within 5% of the ranges recommended by the DNA report and may be ranked efficient if fat is the highest category. An efficient meal rating has all three macro categories (protein, fat, complex carbohydrates) being consumed at a rate within 1-5% of each categories daily percentage. For an efficient meal, the protein, carbohydrates, and fats vary. Regular weekly consumption of efficient meals is an effective way to gradually lose weight, burn fat, and build muscle. If any category has a disparity between it and any other macro category that is larger than 5% it lowers to the next rating of maintenance. A maintenance meal is when fat is the highest percentage of the meal and the differences are within 5% of the recommended ranges. For a maintenance meal, a macronutrient mix is considered average. Regular consumption of maintenance rated meals will likely create a more stable body composition. A maintenance meal rating has all three macro categories (protein, fat, complex carbohydrates) being consumed at a rate within 6-18% of each categories daily percentage as recommended by the DNA report 104. If any category has a disparity between it and any other macro category that is larger than 5% it lowers to the next rating of comfort. A comfort meal is a meal where the difference between percentage of proteins, fats and complex carbohydrates is greater than 18%. A comfort meal is the least efficient macronutrient blend for that individual. Regularly consuming comfort meals will likely result in weight gain. For an active day, the carbohydrate target is increased by 10% and the fat target is decreased by 10% to enable the diet of the patient 106 to provide additional energy and assist with muscle building and fat burn.

TABLE 2
Exemplary Micronutrient Listing of a DNA Report.

| NUTRIENTS | TENDENCY | GOOD SOURCES INCLUDE |
|---|---|---|
| Vitamin A | LOW | Carrots, Kale, Tuna |
| Vitamin B6 | BELOW | Pistachios, Watermelon, Potatoes |
| | AVERAGE | |
| Folate | NORMAL | Pinto Beans, Asparagus, Broccoli |
| Vitamin B12 | LOW | Lean meat, Seafood, Fortified |
| | | Dairy Product |
| Vitamin C | NORMAL | Red Bell Peppers, Strawberries, |
| | | and Oranges |
| Vitamin D | BELOW | Salmon, Egg Yolks, Fortified |
| | AVERAGE | Dairy Milk |
| Vitamin E | NORMAL | Almonds, Spinach, Sweet Potatoes |
| Dietary | INCREASED | Navy Beans, Eggs, Grass Fed Beef, |
| Choline | | Turkey |
| Calcium | NORMAL | Raw Milk, Yogurt, Kale |
| Copper | BELOW | Dark Chocolate, Dried Apricots, |
| | AVERAGE | Sunflower Seeds |
| Iron | ABOVE | Spirulina, Grass Fed Beef, Lentils |
| | AVERAGE | |
| Magnesium | NORMAL | Spinach, Chard, Pumpkin Seeds |
| Phosphorus | ABOVE | Sunflower seeds, Tuna, Turkey, |
| | AVERAGE | Mung Beans |
| Selenium | NORMAL | Brazil Nuts, Yellowfin Tuna, |
| | | Halibut |
| Zinc | ABOVE | Oysters, Toasted Wheat Germ, |
| | AVERAGE | Beef, Pumpkin and Squash Seeds |

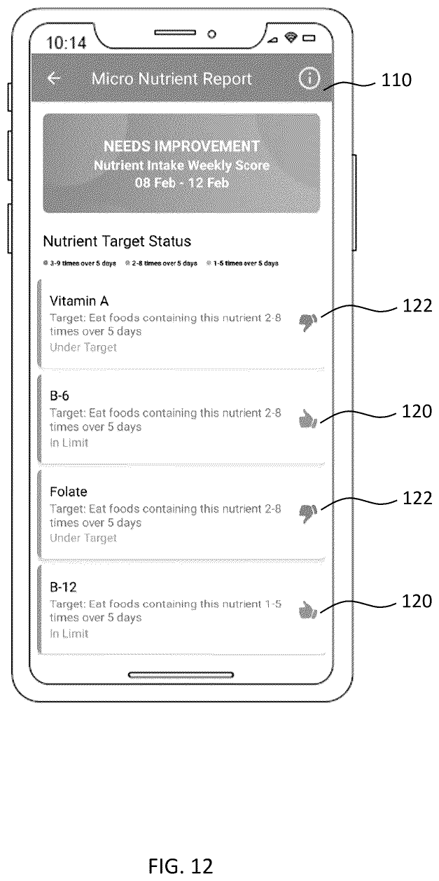

In one embodiment, the micronutrient food score 103 for an individual is calculated for foods showing the list of micronutrients that food contains with the tendency of each nutrient-low, below average, or normal, as illustrated in FIGS. 12-15. The tendency of each patient 106 is genetic and refers to the patient's 106 ability to absorb the nutrients. For patients 106 with low or lower absorption rates for a particular nutrient, the patient 106 may flush out the nutrient before the body has absorbed the nutrient (through urination, for example). The system 100 is operable to target the lesser efficient nutrient tendencies of the patient 106 to create a nutrient balance for the body. The system 100 is operable to receive any DNA report 106 that identifies the absorption tendency of the individual for each of the listed fifteen micronutrients from the report. The system may be adjusted to include additional tendencies or to focus on additional nutrients or other intake metrics. This information is used to propagate specific foods to suggest for the patient 106 to eat. A color-coded system may also be used to assist with patient comprehension. In one embodiment, by operation of the centralized data processing server 102, micronutrient tendencies of a patient 106 are used to create a custom color-coded essential nutrient food list specific to each user. For example, low nutrients may appear in red, below average may appear in orange, and normal may appear in green. Nutrient absorption for patients 106 with increased or above average tendencies may or may not be included in a color coding system depending on the application.

In one embodiment, the system 100 includes reporting of micronutrients within a time period, such as a week or a month, for example. This tracking informs a patient 106 of how well weekly food and nutrient intake aligns with their micronutrient tendencies found in their DNA, allowing the patient 106 to have a snapshot, such as a weekly snapshot, of the nutrients consumed over the previous time period, and raise awareness to those nutrients they may have lacked during the week prior. To aid in patient 106 comprehension, in one embodiment the micronutrient report rates last week's intake by giving a thumbs up 120 (or other indication) when a nutrient has reached a level equal to or greater than, the recommended minimum weekly intake for that nutrient. The patient 106 receives a thumbs down 122 (or other indication) when the recommended intake does not reach their individual recommended weekly total. Recommended intake is relative to each user's genetic tendency for that specific nutrient as everybody patient's intake frequency recommendations are unique to their DNA report. For example, a weekly micronutrient report intake to get a thumbs up are as follows: for a low genetic tendency of a nutrient, the patient should consume a minimum Monday through Friday intake of seven servings of that nutrient; for a below average tendency of a nutrient, the patient should consume a minimum Monday through Friday intake five servings of that nutrient; and for a normal genetic tendency of a nutrient, the patient should consume a minimum Monday through Friday intake of three servings of that nutrient. If a micronutrient weekly intake is less than the above recommended intake, the software application 110 displays a thumbs down for that week and the patient 106 is instructed to target a list 124 of foods containing that nutrient in the upcoming week.

In one embodiment, the centralized data processing server 102 scores foods for each individual patient 106 so each patient 106 can easily identify the healthier foods that contain the nutrients that match their specific average or subpar genetic tendencies. The system 100 generates food scoring to immediately rate how well any food matches the genetic nutrient needs of the user and lowers scores for more processed foods by penalizing foods that contain more fat, sodium or sugar. Additionally, foods that contain the FDA recommended contents of fiber, calcium, iron, and potassium are rewarded with a higher score, regardless of the DNA report 104.

The exemplary calculation below illustrates a user with an exemplary scoring for an avocado for a user with user has low tendency for Vitamin A, a below average tendency for copper, and a normal tendency for Vitamin C.

### Example Micronutrient Deficiency Calculation

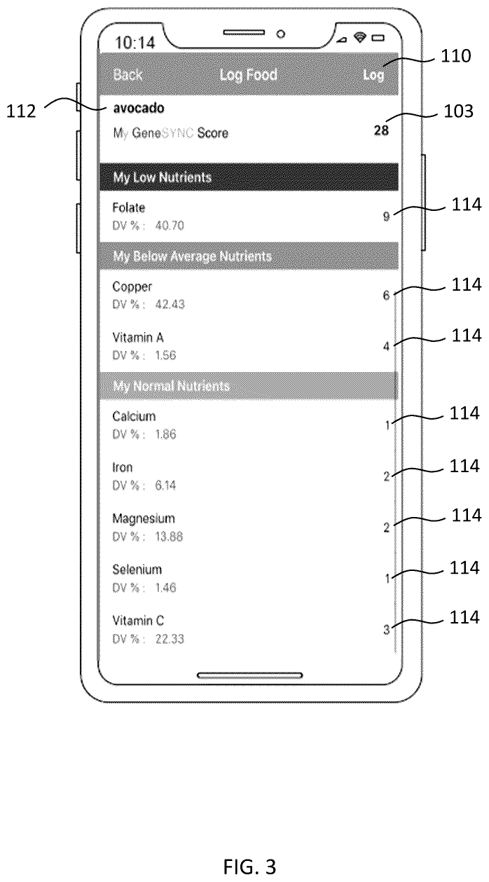

A user has low tendency for Vitamin A, a below average tendency for copper, and a normal tendency for Vitamin C. Using an avocado as an example, a patient 106 will enter the avocado or scan a barcode to upload the information via the software application 110 onto the centralized data processing server 102. By operation of the centralized data processing server 102, if the patient 106 scans the food the system 100 will generate list of nutrients of values of nutrients for the serving using the information contained in the database 108 of foods, such as Nutritionix API. The % dv (daily value) is calculated by dividing the amount of a nutrient in a serving size by its daily value, then multiplying that number by 100. An exemplary points system is included in Table 3 and illustrated in FIGS. 2-3 with points 114.

TABLE 2
Exemplary Food Scoring for Nutrients
Low Absorption of Nutrients

| | | | |
|---|---|---|---|
| <5% | DV | Low Content | 7 Points |
| 6-20% | DV | Medium Content | 8 Points |

| | | |
|---|---|---|
| >20% | High Content | 9 Points |

Below Average Absorption of Nutrients

| | | | |
|---|---|---|---|
| <5% | DV | Low Content | 4 Points |
| 6-20% | DV | Medium Content | 5 Points |

| | | |
|---|---|---|
| >20% | High Content | 6 Points |

Normal Absorption of Nutrients

| | | | |
|---|---|---|---|
| <5% | DV | Low Content | 1 Points |
| 6-20% | DV | Medium Content | 2 Points |

| | | |
|---|---|---|
| >20% | High Content | 3 Points |

The FDA recommended DVs based on a caloric intake of 2,000 calories is: Calcium 1300 mg; Iron 18 mg; Potassium 4700 mg; Vitamin D 20 mcg; Copper 0.9 mg; Folate 400 mcg; Magnesium 420 mg; Vitamin E 15 mg; B-12 2.4 mcg; Phosphorus 1250 mg; Selenium 55 mcg; Vitamin A 900 mcg RAE; B-6 1.7 mcg; Vitamin C 90 mg; and Choline 550 mg.

For example, a patient 106 has low Folate absorption, below average Copper and Vitamin absorption, and normal Vitamin C, Iron, Magnesium, and Selenium absorption. The patient 106 enters an avocado to be consumed. The system 100 generates a score comparing the patient's 106 nutrient absorption to the nutrients of an avocado as stored in the database 108 of nutritional information.

TABLE 3
Scoring of an Avocado for a Patient

| | | | Points |
|---|---|---|---|
| Nutrient | Patent's Level | % DV | (Total 28) |
| Folate | Low | 40.70 | 9 |
| Copper | Below Average | 42.43 | 6 |
| Vitamin A | Below Average | 1.56 | 4 |
| Calcium | Normal | 1.86 | 1 |
| Iron | Normal | 6.14 | 2 |
| Magnesium | Normal | 13.88 | 2 |
| Selenium | Normal | 1.46 | 1 |
| Vitamin C | Normal | 22.33 | 3 |

Tables 4 and 5 illustrate exemplary scoring systems for FDA recommended nutrients and limitations. In some instances, a patient will lose points for eating foods that are low in content or contain unhealthy components.

TABLE 4
Points for FDA Recommended Nutrients
Dietary Fiber

| | | | | |
|---|---|---|---|---|
| <5% | DV | Low Content | −5 | Points |
| 6-20% | DV | Medium Content | 10 | Points |

| | | | |
|---|---|---|---|
| >20% | High Content | 15 | Points |

Vitamin D

| | | | | |
|---|---|---|---|---|
| <5% | DV | Low Content | 3 | Points |
| 6-20% | DV | Medium Content | 6 | Points |

| | | | |
|---|---|---|---|
| >20% | High Content | 9 | Points |

Calcium

| | | | | |
|---|---|---|---|---|
| <5% | DV | Low Content | 3 | Points |
| 6-20% | DV | Medium Content | 6 | Points |

| | | | |
|---|---|---|---|
| >20% | High Content | 9 | Points |

Iron

| | | | | |
|---|---|---|---|---|
| <5% | DV | Low Content | 3 | Points |
| 6-20% | DV | Medium Content | 6 | Points |

| | | | |
|---|---|---|---|
| >20% | High Content | 9 | Points |

Potassium

| | | | | |
|---|---|---|---|---|
| <5% | DV | Low Content | 3 | Points |
| 6-20% | DV | Medium Content | 6 | Points |

| | | | |
|---|---|---|---|
| >20% | High Content | 9 | Points |

TABLE 5
Points for FDA Recommended Limitations
Saturated Fat

| | | | | |
|---|---|---|---|---|
| <5% | DV | Low Content | 5 | Points |
| 6-20% | DV | Medium Content | −10 | Points |

| | | | |
|---|---|---|---|
| >20% | High Content | −15 | Points |

Sodium

| | | | | |
|---|---|---|---|---|
| <5% | DV | Low Content | 5 | Points |
| 6-20% | DV | Medium Content | −10 | Points |

| | | | |
|---|---|---|---|
| >20% | High Content | −15 | Points |

Added Sugars

| | | | | |
|---|---|---|---|---|
| <5% | DV | Low Content | 5 | Points |
| 6-20% | DV | Medium Content | −10 | Points |

| | | | |
|---|---|---|---|
| >20% | High Content | −15 | Points |

In one embodiment, micronutrient which is low must be taken minimum 7 times every 5 days. A micronutrient which is below average must be taken minimum 5 times every 5 days. A micronutrient which is normal must be taken minimum 3 times every 5 days. For example, a patient 106 has below average Vitamin A. This user must eat Vitamin A minimum 5 times from Monday to Friday. If this user has taken in less than 5 times, it is displayed on the software application 110 as under target. If the user eats this nutrient for 5 or more than 5 times, it is displayed on the software application as in target. A weekly report is logged by the software application 110 of every week in program for each nutrient. The system 100 calculates the nutrient score for every week Monday to Friday, for example.

For example, a patient 106 has eaten following foods in this week: Monday: Kale (Vit A, Vit C, Calcium, Magnesium); Tuesday: Chicken (B-6, B-12, magnesium, Phosphorus); Wednesday: Brown Rice (B-6); Thursday: Clams (B-12, Iron, phosphorus); and Friday: Avocado (Folate, Copper, Magnesium). If the DNA report 104 of the patient 106 indicates Vitamin A is below average, the user patient will get 6 points. If the DNA report 104 of the patient 106 indicates Vitamin C is normal, the user will get 3 points. If the DNA report 104 of the patient 106 indicates Calcium is below average, the user will get 6 points. If the DNA report 104 of the patient 106 indicates Magnesium is below average, the user will get 6 points. If the DNA report 104 of the patient 106 indicates Vitamin B-6 is low, the user will get 9 points. If the DNA report 104 of the patient 106 indicates Vitamin B-12 is normal, the user will get 3 points. If the DNA report 104 of the patient 106 indicates Phosphorus is normal, the user will get 3 points. If the DNA report 104 of the patient 106 indicates Iron is normal, the user will get 3 points. If the DNA report 104 of the patient 106 indicates Copper is normal, the user will get 3 points. If the DNA report 104 of the patient 106 indicates Folate is low, the user will get 9 points. Thus, the micronutrient score for the week is calculated: Monday: 6+3+6+6=27; Tuesday: 9+3+6+3=21; Wednesday: 9; Thursday: 3+3+3=9; and Friday: 9+3+6=18 for a total of 84. The software application 110 may also show a list of foods for every particular nutrient which he has consumed in that week.

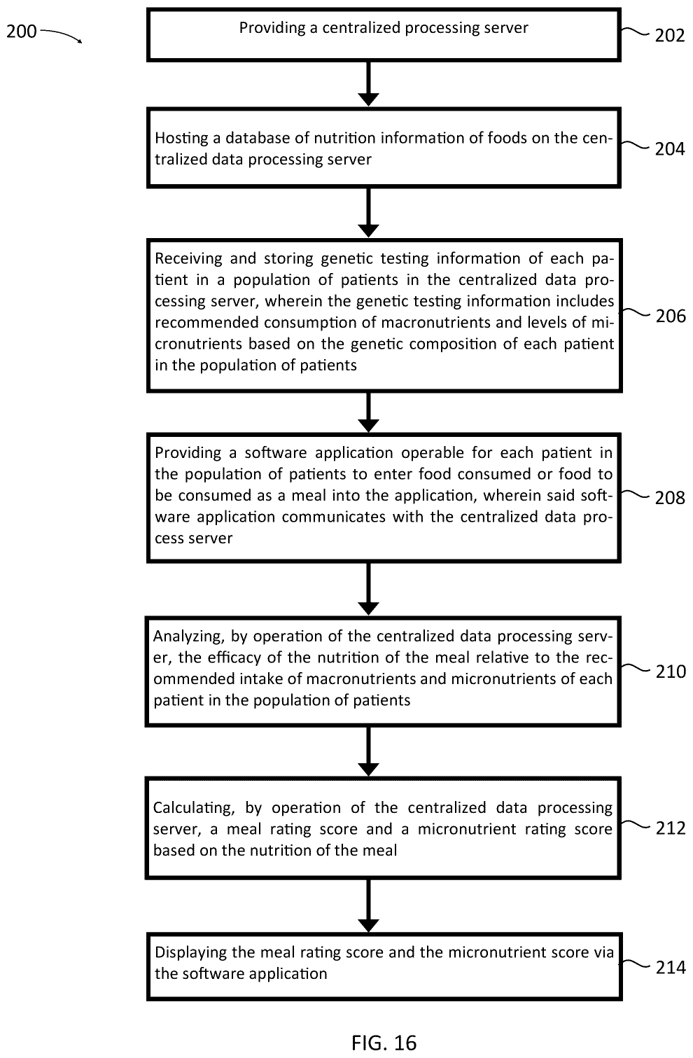

In accordance with embodiments of the invention, a method 200 for food scoring is provided, as illustrated in FIG. 16. The method 200 includes a first step 202 of providing a centralized data processing server 102. The method 200 includes a second step 204 of hosting a database 108 of nutrition information of foods on the centralized data processing server 102. The method 200 includes a third step 206 of receiving and storing genetic testing information 104 of each patient 106 in a population of patients 107 in the centralized data processing server 102, wherein the genetic testing information 104 includes recommended consumption of macronutrients and levels of micronutrients based on the genetic composition of each patient 106 in the population of patients 107. The method 200 includes a fourth step 208 of providing a software application 110 operable for each patient 106 in the population of patients 107 to enter food consumed or food to be consumed as a meal into the application 110. The software application 110 communicates with the centralized data process server 102. The method 200 includes a fifth step 210 of analyzing, by operation of the centralized data processing server 102, the efficacy of the nutrition of the meal relative to the recommended intake of macronutrients and micronutrients of each patient 106 in the population of patients 107. The method 200 includes a sixth step 212 of calculating, by operation of the centralized data processing server 102, a meal rating score 101 and a micronutrient rating score 103 based on the nutrition of the meal. The method 200 includes a seventh step 214 of displaying the meal rating score 101 and the micronutrient score 103 via the software application 110.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A food scoring method, comprising:
providing a centralized data processing server;
hosting a database of nutrition information of foods on the centralized data processing server;
receiving and storing genetic testing information of a patient on the centralized data processing server, wherein the genetic testing information includes recommended consumption of macronutrients and levels of micronutrients based on the genetic composition of the patient;
providing a software application operable for the patient to enter food consumed or food to be consumed as a meal into the application, wherein said software application communicates with the centralized data process server;
analyzing, by operation of the centralized data processing server, of the nutrition of the meal relative to the patient's recommended intake of macronutrients and micronutrients;
calculating, by operation of the centralized data processing server, a meal rating score and a micronutrient rating score based on the nutrition of the meal; and
displaying the meal rating score and the micronutrient score via the software application.

2. The method of claim 1, wherein the meal rating score is calculated based on recommended daily intake of lean protein, complex carbohydrates, and healthy fats.

3. The method of claim 1, wherein the micronutrient rating score is calculated based on the genetic tendencies of the patient for absorptions of micronutrients.

4. The method of claim 1, wherein the micronutrient rating score is assigned a color.

5. The method of claim 3, wherein the micronutrient rating score is 7 for a food containing less than 5% of the daily recommended value of a micronutrient that the patient has a low genetic tendency for absorption, the micronutrient rating score is 8 for a food containing between 6-20% of the daily recommended value of a micronutrient that the patient has a low genetic tendency for absorption, and the micronutrient rating score is 9 for a food containing more than 20% of the daily recommended value of a micronutrient that the patient has a low genetic tendency for absorption.

6. The method of claim 3, wherein the micronutrient rating score is 4 for a food containing less than 5% of the daily recommended value of a micronutrient that the patient has a below average tendency for absorption, the micronutrient rating score is 5 for a food containing between 6-20% of the daily recommended value of a micronutrient that the patient has a below average genetic tendency for absorption, and the micronutrient rating score is 6 for a food containing more than 20% of the daily recommended value of a micronutrient that the patient has a below average genetic tendency for absorption.

7. The method of claim 3, wherein the micronutrient rating score is 1 for a food containing less than 5% of the daily recommended value of a micronutrient that the patient has a normal tendency for absorption, the micronutrient rating score is 2 for a food containing between 6-20% of the daily recommended value of a micronutrient that the patient has a normal genetic tendency for absorption, and the micronutrient rating score is 6 for a food containing more than 20% of the daily recommended value of a micronutrient that the patient has a normal genetic tendency for absorption.

8. The method of claim 1, wherein the micronutrient rating score is calculated based on the recommended daily intake of one or more of Vitamin A, Vitamin B6, Folate, Vitamin B12, Vitamin C, Vitamin D, Vitamin E, Dietary Choline, Calcium, Copper, Iron, Magnesium, Phosphorus, Selenium, and Zinc.

9. The method of claim 1, wherein the software application is operable to scan a barcode to enter food consumed or to be consumed into the application.

10. The method of claim 2, wherein the meal rating score is perfect for a meal containing a blend of macronutrients equal to the recommended consumption of macronutrients for the patient, the meal rating score is efficient for a meal containing a blend of macronutrients within 1-5% of the recommended consumption of macronutrients for the patient; the meal rating score is maintenance for a meal containing a blend of macronutrients within 6-18% of the recommended consumption of macronutrients for the patient, and the meal rating score is comfort for a meal containing a blend of macronutrients greater than 18% outside of the recommended consumption of macronutrients for the patient.

11. A food scoring method, comprising:
providing a centralized data processing server;
hosting a database of nutrition information of foods on the centralized data processing server;
receiving and storing genetic testing information of each patient in a population of patients on the centralized data processing server, wherein the genetic testing information includes recommended consumption of macronutrients and levels of micronutrients based on the genetic composition of each patient in the population of patients;
providing a software application operable for each patient in the population of patients to enter food consumed or food to be consumed as a meal into the application, wherein said software application communicates with the centralized data process server;
analyzing, by operation of the centralized data processing server, the nutrition of the meal relative to the recommended intake of macronutrients and micronutrients of each patient in the population of patients;
calculating, by operation of the centralized data processing server, a meal rating score and a micronutrient rating score for each person in a population of patients based on the nutrition of the meal; and
displaying the meal rating score and the micronutrient score via the software application.

12. The method of claim 11, wherein the meal rating score is calculated based on recommended daily intake of lean protein, complex carbohydrates, and healthy fats.

13. The method of claim 11, wherein the micronutrient rating score is calculated based on recommended daily intake of one or more of Vitamin A, Vitamin B6, Folate, Vitamin B12, Vitamin C, Vitamin D, Vitamin E, Dietary Choline, Calcium, Copper, Iron, Magnesium, Phosphorus, Selenium, and Zinc.

14. The method of claim 12, wherein the meal rating score is perfect for a meal containing a blend of macronutrients equal to the recommended consumption of macronutrients for the patient, the meal rating score is efficient for a meal containing a blend of macronutrients within 1-5% of the recommended consumption of macronutrients for the patient; the meal rating score is maintenance for a meal containing a blend of macronutrients within 6-18% of the recommended consumption of macronutrients for the patient, and the meal rating score is comfort for a meal containing a blend of macronutrients greater than 18% outside of the recommended consumption of macronutrients for the patient.

15. The method of claim 11, wherein the software application is operable to scan a barcode to enter food consumed or to be consumed into the application.

16. A food scoring system, comprising:
a centralized data processing server;
a database of nutrition information of foods;
a software application in communication with the centralized data processing server;
wherein the patient enters food consumed or food to be consumed as a meal into the application;
wherein genetic testing information including recommended consumption of macronutrients and levels of micronutrients based on the genetic composition of the patient is uploaded to the centralized data processing server;
wherein, by operation of the centralized data processing server, the nutrition of the meal relative to the patient's recommended intake of macronutrients and micronutrients is analyzed;
wherein by operation of the centralized data processing server, a meal rating score and a micronutrient rating score are calculated based on the nutrition of the meal; and
wherein the meal rating score and the micronutrient score are displayed via the software application.

17. The system of claim 16, wherein the meal rating score is calculated based on recommended daily intake of lean protein, complex carbohydrates, and healthy fats.

18. The system of claim 16, wherein the micronutrient rating score is calculated based on recommended daily intake of one or more of Vitamin A, Vitamin B6, Folate, Vitamin B12, Vitamin C, Vitamin D, Vitamin E, Dietary Choline, Calcium, Copper, Iron, Magnesium, Phosphorus, Selenium, and Zinc.

19. The system of claim 16, wherein the meal rating score is perfect for a meal containing a blend of macronutrients equal to the recommended consumption of macronutrients for the patient, the meal rating score is efficient for a meal containing a blend of macronutrients within 1-5% of the recommended consumption of macronutrients for the patient; the meal rating score is maintenance for a meal containing a blend of macronutrients within 6-18% of the recommended consumption of macronutrients for the patient, and the meal rating score is comfort for a meal containing a blend of macronutrients greater than 18% outside of the recommended consumption of macronutrients for the patient.

20. The system of claim 16, wherein the software application is operable to scan a barcode to enter food consumed or to be consumed into the application.
